(19) United States
(12) Patent Application Publication
(10) Pub. No.: US 2024/0077484 A1
(43) Pub. Date: Mar. 7, 2024

(54) Addressable Microfluidics Systems and Methods for In Vivo Applications

(71) Applicant: New Jersey Institute of Technology, Newark, NJ (US)

(72) Inventors: Roman Voronov, Oklahoma, OK (US); Anh Tong, Newark, NJ (US)

(73) Assignee: New Jersey Institute of Technology, Newark, NJ (US)

(21) Appl. No.: 18/508,563

(22) Filed: Nov. 14, 2023

Related U.S. Application Data
(63) Continuation-in-part of application No. 17/072,854, filed on Oct. 16, 2020.
(60) Provisional application No. 63/425,168, filed on Nov. 14, 2022.
(60) Provisional application No. 62/916,586, filed on Oct. 17, 2019.

(51) Int. Cl. G01N 33/573; A61B 10/00; B01L 3/00; G01N 33/543; G01N 33/68

(52) U.S. Cl. CPC G01N 33/573; A61B 10/0045; B01L 3/502738; G01N 33/54386; G01N 33/6887; A61B 2010/009; B01L 2200/12; B01L 2300/023; B01L 2300/123; B01L 2400/06; G01N 2333/78; G01N 2333/90206

(57) ABSTRACT

A microfluidic device capable of performing nondisruptive fluid manipulations in a living host is provided. The microfluidic device may include a combinatorial multiplexer for better scaling of multiple time points and biological signal measurements. The collected samples may be transported, stored and analyzed ex vivo for analytical ease and flexibility, e.g., by a sample analysis assay chip. The microfluidics device may include structure for maintaining fluid equilibrium within the host during the sampling to avoid damage to the host or to the implant.

### CROSS-REFERENCE TO RELATED APPLICATIONS

The present application claims priority benefit to a provisional patent application entitled “Addressable Microfluidics Technology,” which was filed on Nov. 14, 2022, and assigned Ser. No. 63/425,168. The present application also claims priority benefit to a co-pending non-provisional patent application entitled “Automated Addressable Microfluidic Technology for Minimally Disruptive Manipulation of Cells and Fluids within Living Cultures,” which was filed on Oct. 16, 2020, and assigned Ser. No. 17/072,854, and which application claims priority to a provisional application filed on Oct. 17, 2019, and assigned Ser. No. 62/916,586. The entire contents of the foregoing provisional applications and non-provisional application are incorporated herein by reference.

### STATEMENT REGARDING FEDERALLY SPONSORED RESEARCH

This invention was made with government support under Agreement No. UL1TR003017 awarded by the National Center for Advancing Translational Sciences (NCATS), a component of the National Institute of Health (NIH). The government has certain rights in the invention.

### FIELD OF THE DISCLOSURE

The present disclosure relates to a cell-culturing microfluidic system with improved addressable microfluidic technology for non-destructive analysis of materials implanted in vivo.

### BACKGROUND

Custom materials are commonly developed for implantation purposes, such as engineered tissue scaffolds and drug delivery vehicles (e.g., an anti-cancer drug emitting material implanted near a tumor). FDA approval for such implants could take 10-15 years [Brown, D. G., et al., Clinical development times for innovative drugs. Nature reviews. Drug discovery, 2021] and cost upwards of a billion US dollars. [Wouters, O. J., M. McKee, and J. Luyten, Estimated research and development investment needed to bring a new medicine to market, 2009-2018. Jama, 2020. 323(9): p. 844-853]. Consequently, failure, which can be as high as 90%) [Mullard, A., Parsing clinical success rates. Nature Reviews Drug Discovery, 2016. 15(7): p. 447-448.], at any stage of the trials results in enormous financial losses that are often responsible for many biomedical companies going out of business or moving their operations abroad. Among the preliminary determinations, accurate forecasting of the materials' performance and safety in vivo is crucial to making an informed decision as to whether it makes sense to commit financial resources to the clinical trials. Hence, animal studies constitute the last major testing step prior to performing the trials in human patients. [Webber, M. J., et al., A perspective on the clinical translation of scaffolds for tissue engineering. Annals of biomedical engineering, 2015. 43(3): p. 641-656] As such, animal studies serve as the most physiologically representative predictor of the biomaterial's safety and efficacy, once a material is implanted into a human.

However, the in vivo performance of an implanted material is notoriously difficult to analyze without explanting the sample from the animal first. In most cases, this also means euthanizing the test subject (e.g., recovering a scaffold from a cranial defect requires non-survival surgery). [Spicer, P. P., et al., Evaluation of bone regeneration using the rat critical size calvarial defect. Nature Protocols, 2012. 7(10): p. 1918-1929] Furthermore, the subsequent analysis is typically limited to just 2-3 signals per retrieved sample, because microscopy dyes overlap in color, and many assays are incompatible with each other. Lastly, critical spatial information, such as tissue induction (i.e., areas of vascular in-growth and cell migration from the host into the implant) and necrosis (i.e., areas of cell death due to limited nutrient supply and/or generation of excess waste products), may be distorted by the explantation surgery and/or histology embedding/slicing. All this significantly increases experimental costs and results in data discontinuity because a new animal is required to obtain each different time point (see, e.g., FIG. 1A, where testing engineered bone in three different animals is taken as an example). Such inconsistency can lead to misleading results. Thus, the sacrificial analysis both slows down the advancement of knowledge and bottlenecks the translation of novel biomedical innovations to the clinical setting. Hence, there is a need for a technology capable of continuous monitoring within the same individual, and ideally capable of probing at separate locations within the implant non-disruptively. There is a further need that a technology provide continuous spatiotemporal information that is robust and that would better inform researchers and/or companies regarding whether it makes sense to proceed with human trials. These and other needs are met by the systems and methods disclosed herein.

### SUMMARY

A microfluidic platform is disclosed herein that supports and facilitates performance of non-disruptive fluid manipulations within applicable spatial constraints. The disclosed addressable microfluidic plumbing is specifically adapted for in vivo implantation and may be integrated with a combinatorial multiplexer, for better scaling of many time points and/and or biological signal measurements. The collected samples may be transported, stored and analyzed ex vivo, thereby significantly enhancing ease and flexibility of testing modalities. Beyond bone tissue engineering applications, the disclosed microfluidic platform may be employed in various in vivo sampling applications. The successful outcomes of its advancement will benefit companies developing, testing, and producing vaccines and drugs, by accelerating the translation of advanced cell culturing tech to the clinical market. Moreover, the nondestructive monitoring of the in vivo environment will lower animal experiment costs and provide data-gathering continuity superior to the conventional destructive analysis. Lastly, the reduction of sacrifices stemming from the use of this technology will make future animal experiments more ethical.

In accordance with embodiments of the present disclosure, an exemplary microfluidic platform is disclosed. In one embodiment, an exemplary process includes implanting a microfluidics device with an intergrated biomaterial scaffold into a host (e.g., a living test animal). Although different applications are possible, in this embodiment a goal is to evaluate the biomaterial's ability to serve as an artificial bone implant. Therefore, it may be inserted into a defect in the animal's skull that has been surgically created to be too large to heal on its own (i.e., without the biomaterial scaffold filling the gap in the animal's bone).

The microfluidic device is then connected to an automated external pumping system via microfluidics tubing. In one embodiment, a ‘multiplexer’ (i.e., a non-implantable microfluidics device) could be used to reduce the external hardware requirement for running the implant). Subsequently, fluidic and/or cellular probings are programmed to be collected at different times and locations on the biomaterial's surface. This creates a stream of spatiotemporal data about the integration of the scaffold into the host's native bone tissues, without the need for sacrificing the animal for each single time point (as would be typically required by the conventional analysis approaches).

Optionally, the microfluidics device can also be used to deliver (as opposed to collecting) drugs, cells and chemicals to help to induce tissue regeneration. In other words, it can be used to assist the biomaterial in its job of healing the impacted bone (or other tissues in different applications).

Operations of the device (e.g., both the sampling and the optional delivery of cells and chemicals) can be monitored and controlled automatically via a programable controller, thereby reducing human labor that would have been needed to perform a similar experiment.

Furthermore, once the collected fluid and/or cells samples are transported out of the host, they can be optionally stored in an assay analysis chip for an automated analysis and for real-time feedback to the computer controller that is monitoring (and optionally, assisting) the implant.

The external pumping system can be optionally miniaturized to be worn by the animal on its back, so that the test subject would have more freedom to move around its cage (as opposed to being constantly tied down to external pumping hardware via tubing).

In addition, at the end of an experiment, the scaffold may be explanted for end-point validation of the results. Alternatively, the animal may be allowed to live out the remainder of its life with the device implanted (but disconnected from the sampling hardware). Either way, the disclosed modality offers a reduction in the number of animal sacrifices required to collect desired information, e.g., evaluate initial and intermediate time points (e.g., weeks 4 and 8 in FIG. 1A) with ethical benefits.

In one embodiment, an in vivo addressable microfluidic device may include a control valves layer, a flexible membrane, a payload/probing plumbing layer with addressable ports (e.g., openings connected to channels and operated by valves), a sampling chambers layer, and a scaffold (e.g., a poly-L-lactic acid (PLLA) scaffold). The sampling chamber may serve to isolate the plumbing of the microfluidic device from fluid(s) of a host's body. The sampling chamber may be used to maintain fluid equilibrium and prevent contamination from surrounding tissues.

In one embodiment, the control valves layer may serve to inflate the flexible membrane to block/re-route fluid flow at desired locations within the payload/probing plumbing layer. The flexible membrane may serve to obstruct fluid flow at desired locations within the payload/probing plumbing layer upon being inflated. The payload/probing plumbing layer may be provided to flow chemical and/or cell payloads to/from desired locations within the device, and such payload/probing plumbing layer may cooperate with the control valves layer and/or with the flexible membrane to achieve this purpose. The sampling chambers layer generally serves to collect cell and fluid samples for ex situ analysis of the implant's performance in the host's body. The scaffold (e.g., a PLLA scaffold) is an example biomaterial implant that may be implemented according to the disclosed system and method, although in other embodiments an implant that takes different forms and/or that is fabricated, in whole or in part, from different material(s), may be employed.

In one embodiment, an implantable microfluidics device may include control valves layer, flexible membrane, payload/probing plumbing layer, sampling chambers layer and a biomaterial scaffold that is being tested in vivo. Collectively, this assembly is termed as the ‘addressable implant’ in FIG. 10A.

In one embodiment, the disclosed system may include a ‘multiplexer’ (i.e., microfluidics device that reduces the external hardware requirement for running the ‘addressable implant’) and the ‘sample analysis assay chip’ (i.e., a microfluidics device that is used for ex situ analysis of samples obtained from in vivo. Both the ‘multiplexer’ and the ‘sample analysis assay chip’ are shown in FIG. 10a.

In one embodiment, the microfluidic plumbing system may include an array of addressable microfluidic ports that may serve as access openings, connecting fluid flow channels to different locations within one or more cell culture chambers. The system may include an inflatable on-chip valve layer that includes inflatable on-chip valve(s), wherein the inflatable on-chip valve(s) can be actuated to open or closed states, which allow or block fluid flow to the addressable microfluidic ports. In this way, fluid flow can be directed to any desired location in the one or more cell culture chambers.

The system may include one or more flexible membrane layers disposed below the valve layers and one or more fluid flow layers disposed below the flexible membrane layers, wherein the addressable port openings are disposed within the one or more fluid flow layers to define a path for payloads carried by the fluid flow. The cell culture chamber may include one or more cell layers and the cell culture chamber may be located below one or more fluid flow layers. Thus, in exemplary embodiments, a method for non-destructive in vivo analysis of implanted materials is provided that includes (i) inserting a microfluidics device within a living organism, (ii) collecting samples from the living organism at a plurality of distinct targeted locations, wherein the samples include fluid samples, biological samples or both fluid and biological samples, and (iii) exporting the collected samples out of the living organism for analysis ex vivo. The samples may be collected continuously over time, and may be collected at pre-set intervals

In exemplary embodiments, the microfluidics device includes (i) a control valves layer, (ii) a flexible membrane positioned beneath the control valve layer, (iii) a payload plumbing layer with customizable addressable ports under the flexible membrane, and (iv) a layer of chambers beneath the payload plumbing layer from which the samples are collected.

The microfluidics device may include at least one programmable addressable valve capable of regulating fluid flow. The microfluidics device may include an integrated multiplexer. The integrated multiplexer may be configured to function as a fluidic switchboard by directing flow to and from the microfluidic device. The integrated multiplexer may direct flow to and from the microfluidics device based on analytical need. The integrated multiplexer may reduce external hardware requirements for analysis of the samples. The microfluidics device may include an integrated analysis chip that processes the collected samples.

The microfluidics device may be configured and dimensioned for insertion into a cranial bone defect.

The method for non-destructive in vivo analysis of implanted materials may include removal of the microfluidics device from the host organism for post-analysis.

The sampling chambers layer may be configured to isolate the microfluidics device from surrounding bodily fluids of the living organism.

The microfluidics device may include means for maintaining fluid equilibrium within the living organism during sample collection.

The method for non-destructive in vivo analysis of implanted materials may use the ex vivo analysis of the samples to monitor osteogenesis within the living organism.

The method for non-destructive in vivo analysis of implanted materials may include integrating the microfluidics device with a structural scaffold fabricated at least in part from a material being tested. The microfluidics device may be attached to the structural scaffold by a central fastening mechanism. The microfluidics device may defines a curvature to conform to one or more anatomical structures of the living organism.

The method for non-destructive in vivo analysis of implanted materials may include leveling the microfluidics device when implanted in the living organism.

The sampling chambers layer may include an outer circular boundary and an inner section divided into multiple segments for distinct sampling. The sample analysis may be conducted using an assay chip.

In exemplary embodiments, a microfluidics system is provided that includes (i) a control valves layer; (ii) a flexible membrane beneath the control valve layer; (iii) a payload plumbing layer with configurable addressable ports beneath the flexible membrane; (iv) a sampling chambers layer beneath the payload plumbing layer; (v) an integrated multiplexer for fluid control; and (vi) an integrated analysis chip for sample analysis.

The one or more of the control valves layer, the payload plumbing layer and the sampling chambers layer may be independently prepared and then joined together with the flexible membrane. The one or more of the control valves layer, the payload plumbing layer and the sampling chambers layer may be fabricated by lithography, etching, machining laser-cutting, 3-D printing, and combinations thereof.

The control valves layer, the payload plumbing layer, the sampling chambers layer and the flexible membrane may be joined together by a bonding method that involves chemical bonding, thermal bonding, pressure bonding, and combinations thereof.

The control valves layer, the payload plumbing layer and the sampling chambers layer may be manufactured in their entireties by 3-D printing, sacrificial template gel-casting, or a combination thereof.

The microfluidics system may include a system for analysis of biological processes that includes (i) a data processing unit configured to analyze data obtained from collected samples; and communication means for transmitting data from the microfluidics device to the data processing unit.

The microfluidics system may include a plurality of chemical assays tailored for analyzing samples gathered in vivo. The plurality of chemical assays may include one or more assays for analyzing cranial bone defect-related properties.

The control valves layer, the flexible membrane, the payload plumbing layer, the sampling chambers layer, the integrated multiplexer, and the integrated analysis chip, may be miniaturized and integrated into a single portable system.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE FIGURES

To assist those of skill in the art in making and using the disclosed addressable microfluidic technology and associated systems and methods, reference is made to the accompanying figures, wherein:

FIG. 1A shows a conventional scaffold testing in vivo process. Typically, scaffolds are implanted into multiple animals, each of which is then sacrificed at various times to provide a trajectory of how the biomaterial induces bone generation in vivo.

FIG. 1B illustrates an exemplary microfluidic process, in accordance with one embodiment of the present disclosure. An implanted device continuously provides real-time cell and fluid sampling measurements of bone formation from within the same living animal. This approach results in cheaper, continuous, and reliable data obtained much faster and using less animals.

FIG. 2 shows a schematic view of a device/system according to an embodiment of the present disclosure. As shown at the left of FIG. 2, a lab rodent is schematically depicted with a material-testing device implanted into a circular critical sized defect in its skull. As shown at the right of FIG. 2, a side view of a microfluidics device according to an embodiment of the present disclosure is schematically depicted, wherein the microfluidics device uses addressable ports to probe cells and fluids at targeted locations within the implant. When a valve is inflated, the associated port is closed off; but when the valve is not actuated, negative pressure is used to collect a micro-“biopsy” for ex situ analysis;

FIGS. 3A and 3B are diagrams showing how the Payload/Probing Plumbing multilayered mold and the PDMS layers are stacked on top of each other in an embodiment of the present disclosure. In FIG. 3A, the master mold for payload/probing plumbing layer (with addressable ports) consists of two sub-layers: a round profile flow channels and addressable microfluidics ports on top of the round channels. In FIG. 3B, an in vivo addressable microfluidic device is provided by stacking five different 12 mm diameter PDMS layers and an 8 mm diameter PLLA scaffold on top of each other. In FIG. 3B, reference #1 corresponds to a Control Valves Layer; reference #2 corresponds to a 35 μm Flexible Membrane; reference #3 corresponds to a Payload/Probing Plumbing Layer (with addressable ports); reference #4 corresponds to a Sampling Chambers Layer; and reference #5 corresponds to a 8 mm diameter PLLA Scaffold;

FIGS. 4A-4E show an exemplary addressable microfluidics device for non-destructive analysis of biomaterial implants in vivo according to the present disclosure. In FIG. 4A, the left-hand side shows a photograph of an exemplary device's size relative to a US penny coin. It is noted that the tubing may be miniaturized as compared to the prototype system shown in FIG. 4A. The right-hand side of FIG. 4A provides a close-up top view of a device that has an overall diameter of 12 mm. Note, that the tubing is omitted from this view for clarity. Also note that since the PLLA scaffold that goes below the device is optically opaque, it is also omitted for clarity. In FIG. 4B, a Top-view Diagram of an exemplary microfluidics plumbing design is provided showing its circularity. In FIG. 4C, a side-view diagram is provided showing how an exemplary device is joined with an 8 mm PLLA scaffold via a center bolt In FIG. 4D, a photograph is provided showing how an exemplary PDMS part of a device is joined with an 8 mm PLLA scaffold via a center bolt. Of note, the two parts have not been pressed towards each other to show an early point in assembly. Also, the nut at the top is omitted to demonstrate a visible spacing between the PDMS and the PLLA. In FIG. 4E, a photograph of a stacked device is provided, with the PDMS and the PLLA parts fully pressed against each other using a nut at the top. Of note, a flat head bolt is used in

FIGS. 4D and 4E to minimize protrusions into the intracranial space of the animal (as shown below the PLLA scaffold).

FIGS. 5A and 5B are views of a circularly curved addressable microfluidic plumbing for in vivo implantation. In FIG. 5A, a top view schematic of the Control Valves (O-shaped) and the Payload/Probing Plumbing (arch-shaped) layers are provided, aligned on top of each other. The black cross is an alignment mark, which also designates placement of a screw that may be used to attach a biomaterial scaffold below the device. In FIG. 5B, a microscopy image (top view) of a fabricated microfluidic design as shown in FIG. 5A is provided.

FIGS. 6A-6D show an addressable fluid manipulation sequence in an implantable device (top views) according to the present disclosure. In FIG. 6A, a diagram of delivery of a dye to the top left corner port in the first/top curved address row is shown. In FIG. 6B, a diagram of sampling of the delivered dye via a neighboring port in the second curved address row is shown. The black cross is an alignment mark showing where an attachment member, e.g., a bolt0 may be inserted. FIGS. 6C and 6D are microscopy equivalents of FIGS. 6A and 6B.

FIGS. 7A-7C show views illustrating isolation of an addressable microfluidic implant from a host's tissues and pressure rebalancing via a Sampling Chambers Layer according to an embodiment of the present disclosure. In FIG. 7A, a diagram is provided showing how the device's various layers may be leveled when implanted in vivo. The Sampling Chambers Layer is between the PDMS and the PLLA Scaffold, surrounded by the Periosteum. Since the former is wider than the Scaffold, the layer's outer edge sits directly on top of an animal's scull and blocks any fluid and/or cell transport in the radial directions. Also shown is the fluid replenishment (darker arrows) that occurs in tandem with the sampling (lighter arrows) to maintain a pressure balance. The attachment member (e.g., screw) is omitted for clarity. In FIG. 7B, a microscopy image (top view) is provided that uses food dyes to highlight the device's “pizza slice”—shaped chambers, Control Valves (O-shaped) and the Payload/Probing Plumbing (straight and bypass channels). In FIG. 7C, the image at the left is a diagram of Sampling Chambers Layer showing its walls in a darker shade and the void space from which the probings can be taken in a lighter shade. In the center/right of FIG. 7C, a zoom in of a “pizza slice”—shaped chamber is provided that shows two addressable ports working in tandem to quench any positive or negative pressure excess build-up: when one port is probing samples (with “up” arrow), the other one is open for the media replenishment to flow in (with “down” arrow), and vice versa.

FIG. 8 shows a three panel diagram of an exemplary implanted biomaterial-testing device's operation according to the present disclosure. In the left panel of FIG. 8, a storage bottle is shown from which fresh culture media and maintenance drugs may be replenished. In the center panel of FIG. 8, a diagram is provided showing the microfluidic device receiving replenishment media from the storage bottle, as it is sampling the in vivo environment above the PLLA scaffold implant and sending off the results to the Sample Analysis Assay Chip (SAAC). In the right panel of FIG. 8, a diagram is provided showing a possible iteration of the SAAC, in which its matrix of wells consists of four rows (e.g., to match the four rows of Payload/Probing Plumbing channels in FIGS. 5A-5B, and in FIGS. 6A-6D). Of note, the number of columns in the matrix remains a free parameter that could be determined, for example, based on the number of the desired time points, locations and signals that the user wants to measure.

FIGS. 9A and 9B shows an exemplary Sample Analysis Assay Chip (SAAC) (top view) according to the present disclosure. In FIG. 9A, a diagram of a 4×8 array of addressable microfluidics sample analysis wells is provided. In FIG. 9B, a microscopy image of a fabricated 4×8 Sample Analysis Chip is provided. Food dyes are used to highlight the addressable circular 0-shaped valves and the sample payload channels and storage wells (inside of the valves). The latter are shown at different stages of being filled (i.e., fully, partially, or not at all). In exemplary embodiments, each well has a 20 μL volume.

FIGS. 10A and 10B show an exemplary plumbing setup for an addressable microfluidic implant and two chips supporting it, according to the present disclosure. In FIG. 10A, a diagram showing the plumbing setup is provided, with all three microfluidic systems working together in unison: the Combinatorial Multiplexer actuating the Addressable Implant and the SAAC. Vertical junctions #1-4 of the chip are dedicated for the inputs of the payload/probing plumbing on the Addressable Implant; Junctions #5-8 are dedicated for the control valves on the same chip. Junctions #9-16 actuate the control valves on the SAAC; and then the output of the SAAC is actuated with negative pressure from Junctions #17-20. Horizontal control lines #4 and #6-8 of the multiplexer are actively blocking all lines, except for the right arm of Junction #1. This enables flow through the top row of the Addressable Implant. In FIG. 10B, a microscopy image of a fabricated system with food color highlights is provided. The implantable device is in a dish. Of note, although a combinatorial multiplexer consisting of 8 control lines (horizontal) can operate up to 70 flow lines (vertical), only a portion of the latter that is needed to run the system is shown.

FIG. 11 shows a schematic view of a system according to the present disclosure. In the left pane, a lab rodent with a biomaterial-testing device implanted into a critical sized defect (circular) in its skull is schematically depicted. All of the electronics pressurizing and driving the device, as well as the supporting microfluidics chips that perform the multiplexing and the analysis, are fitted into a “backpack” worn by the rodent. In the right panel, a side view of a microfluidics device that uses the addressable ports to probe cells and fluids at targeted locations within the implant is provided. When the black valve is inflated, the port is closed off; but when the valve is not actuated, negative pressure is used to collect a micro-“biopsy” and send it off for ex situ analysis,

### DETAILED DESCRIPTION

Reference will now be made in detail to certain exemplary embodiments according to the present disclosure, certain examples of which are illustrated in the accompanying drawings.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein “tissue scaffold” may refer to any porous or highly porous scaffold bio-compatible material, which may act as a template for tissue regeneration or cell culturing experiments, to guide the growth of new tissue or the cell distribution in the 3D culture.
As used herein “microfluidics” may refer to any device or devices that use micro-sized channels. connected to a pumping source, for delivery or sampling of fluids within artificial biological environments. Such devices may be used for cell culture experiments mimicking tissue environments, biomanufacturing, disease modeling, toxicity testing, and the like.

As used herein, the term “compartment” can refer to a physically delineated space, such as a space between walls and/or housing (e.g., a channel through a membrane), or it can refer to an area or region in space (e.g., a position in a cell culture). For example, a compartment may refer to a region in space positioned within a three-dimensional microfluidic scaffold structure or a position between or proximal to valves. In some instances, the terms “port” and “compartment” can be used interchangeably because these terms can refer to an area in space relative to the position of a valve, wherein one of skill in the art (in view of the present disclosure) would understand how to arrange and manipulate valves and microfluidic plumbing to deliver a payload to one or more desired compartments depending upon the particular configuration of the microfluidic system. One of skill in the art would immediate envisage, in view of the present disclosure, the meaning and scope of a compartment and ports as used herein.
Various aspects of the present disclosure relate to microfluidic systems, including cell cultures and 3D microfluidic tissue scaffolds, that further include addressable plumbing (i.e., channels, valves, ports, chambers, and the like). For example, an exemplary 3D addressable system according to aspects of the present disclosure may comprise a combination of channels, valves, ports, and/or chambers. In some embodiments, chambers without a bottom floor wall may be referred to as a port. In an exemplary embodiment, a microfluidic system or microfluidic scaffold may comprise a plumbing system comprising addressable microfluidic valves. A plumbing system may also comprise a pumping system. The combination of pumping system and valves (both on-chip and off-chip) may be used to deliver or draw fluid (e.g., cell culture media containing a biological agonist) and/or cells to/from each address (e.g., location of interest in an array of addressable chambers, ports, valves, etc.) by actuating the corresponding address (i.e., port). In some embodiments, the actuation may be achieved via an on-chip valve, or valves that may either guide the fluid's (which could be carrying a chemical and/or cells payload) path to or from a port, or may route the payload to bypass the port. In some embodiments, the on-chip valves may comprise on-chip microfluidic valves that are driven by off-chip (i.e. external) solenoid valves.

According to aspects of the present disclosure, the disclosed addressable microfluidic plumbing systems may be configured to enable targeted fluid and/or cell delivery and/or removal (including collecting for sampling) at targets (i.e., precise locations) within a tissue scaffold or a cell culturing device, and with a high spatial resolution. Some exemplary microfluidic system embodiments disclosed herein may include addressable ports at points of interest (i.e., select points where either chemical delivery and/or sampling may be performed in a microfluidic scaffold or device).

Addressable microfluidic plumbing systems comprising valves, chambers, ports, and/or supply channels may permit a microfluidic cell culturing device (e.g., 3D microfluidic tissue scaffold) to monitor and/or control cell behavior at a targeted locations within the device, or in a tissue scaffold, for the purposes of, but not limited to: regenerative medicine, tissue engineering, drug discovery, wound assays and biomedical devices.

In regenerative medicine and other applications, the microfluidic addressable valves disclosed herein may assist in tissue patterning by selective delivery of cell differentiation factors and other agonists to different parts of a tissue scaffold. The disclosed addressable microfluidic ports may also manipulate cell behavior via localized chemo-signal delivery. The microfluidic valves may also be used for non-invasive cell and tissue development monitoring by performing localized chemical probing (e.g., for ex-situ sacrificial assays) within the tissue scaffold during culturing, and without sacrificing the sample or disrupting the experiment. Lastly, the microfluidic valves may be useful in adaptive culturing control with real-time feedback (e.g. adjusting culturing conditions based of observed cell behavior and/or tissue development) by combining cell control with monitoring.
According to aspects of the present disclosure, microfluidic addressable plumbing (e.g., ports, chambers, flexible valves, etc.) as disclosed herein may also be used for cell development during drug discovery. In other words, a response, or responses, to drugs may be monitored in real-time by delivering different doses of drugs to various locations within a colony of cultured cells or in a living tissue.
Biomedical devices can incorporate the disclosed microfluidic addressable plumbing to operate as an organ-on-a-chip device, and may be used for drug development, diseases modeling, and/or personalized medicine—where spatial chemical delivery or sampling may be necessary.

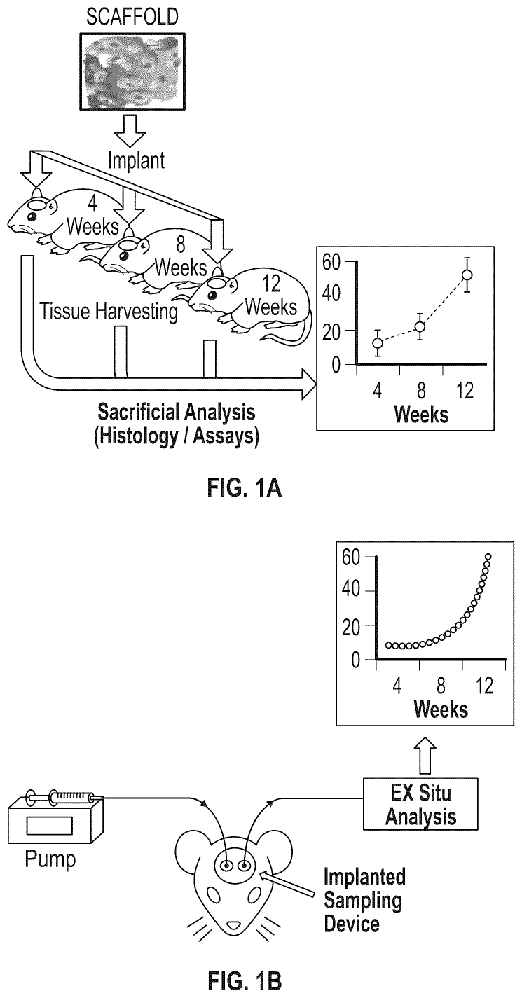

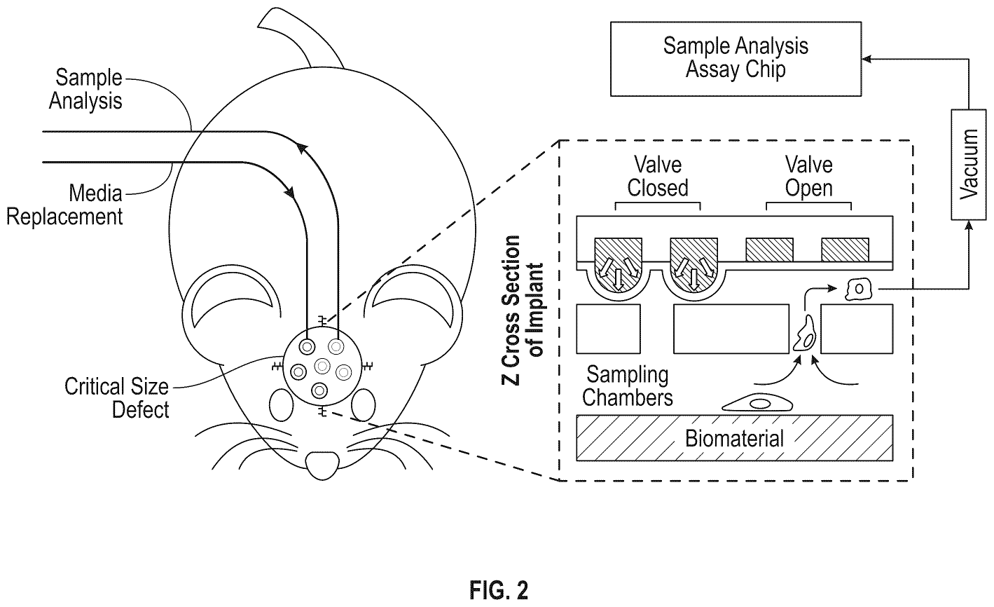

With initial reference to FIG. 1B, an exemplary microfluidic process for an engineered evaluation is shown. As shown in FIG. 1B, in this process, only a single animal is required to obtain all time points from weeks 4 to 12 of a hypothetical experiment. In this embodiment, an implanted device continuously provides real-time cell and fluid sampling measurements of a bone formation from within the same living animal. This results in less expensive, continuous, and reliable data obtained much faster and using less animals, as compared to the conventional method shown in FIG. 1A.

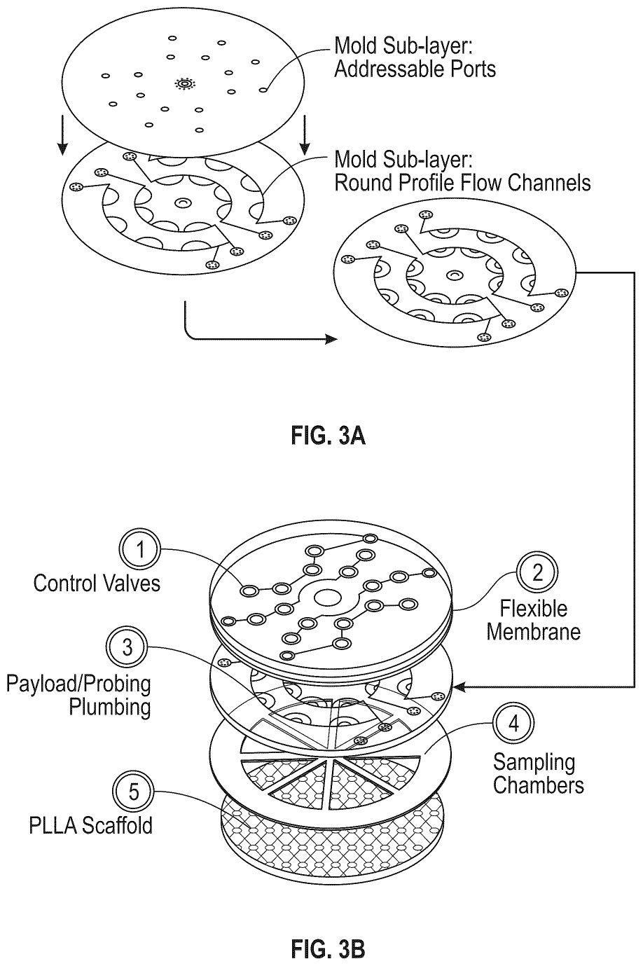

FIG. 2 illustrates an overview of one embodiment of the disclosed microfluidics technology. The left side of FIG. 2 shows a lab rodent with a biomaterial-testing device implanted into a critical sized defect in its skull. The right side of FIG. 2 is a side view of the microfluidics device that uses the addressable ports to probe cells and fluids at targeted locations within the implant. When the valve is inflated, the port is closed off; but when the valve is not actuated, negative pressure is used to collect a micro-“biopsy” and send it off for ex situ analysis.

To demonstrate the microfluidics technology's capabilities, its compatibility with a critical size calvarial defect has been demonstrated—a “gold standard” model to test engineered bone tissue scaffolds in living rats. Specifically, in this model, a round hole is created in the rodent's skull, larger than what can be healed naturally (8 mm diameter for a rat). [See, Cooper, G. M., et al., Testing the “critical-size” in calvarial bone defects: revisiting the concept of a critical-sized defect (CSD). Plastic and reconstructive surgery, 2010. 125(6): p. 1685]

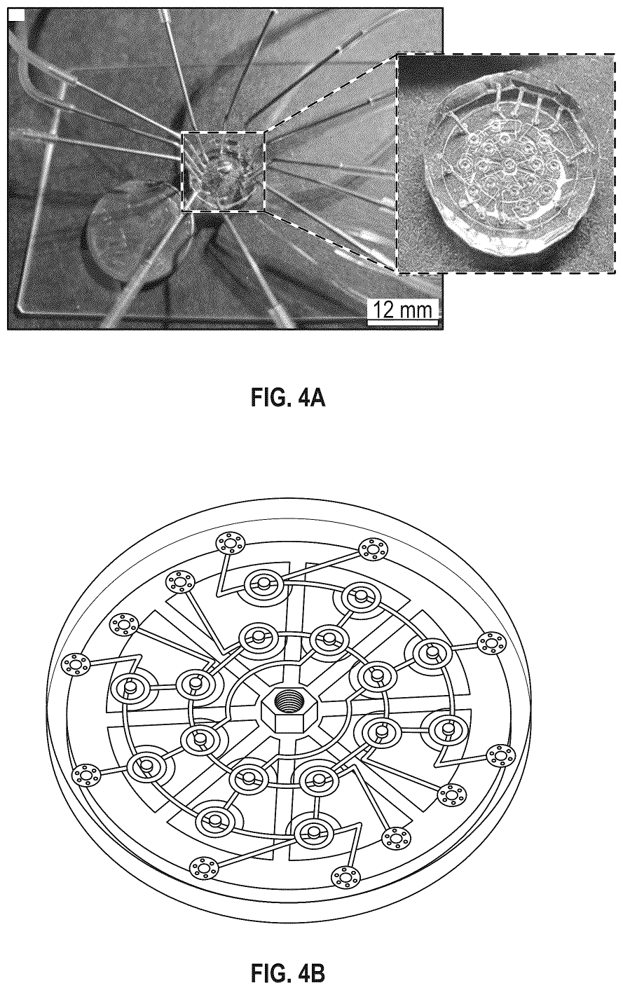

The defect is then filled with a scaffold that is intended to assist the host to heal the injured bone (see the circular disk in FIG. 2, left pane) better than it would have without the intervention. However, in the present embodiment, the implanted biomaterial is accompanied by a microfluidics plumbing (see FIG. 2, right pane). An exemplary microfluidics plumbing design is shown in FIGS. 3A and 3B.

Specifically, once the device is implanted, the host's cells infiltrate the implanted biomaterial that serves as the “floor” of the structure (see FIG. 2, right pane). Vacuum may then be applied to the microfluidics ports at the top of the device to collect micro biopsies from specific locations within the “Sampling Chambers” space (FIG. 2, right pane, and FIGS. 3A and 3B) and transport the collected probings for ex-situ analysis (thereby overcoming the need to explant the entire scaffold). The fluid samples would then be gathered on an external Sample Analysis Assay Chip (SAAC), as described below. From here, they would be analyzed by running assays in a separate microfluidics device, as described below; after which, the bone regeneration results could be recorded by a lab technician or automatically by a computer.

One embodiment of an exemplary sampling technology developed for the non-destructive analysis of biomaterial implants in vivo via automated minimally disruptive manipulations of fluids in an implanted microfluidics device is provided. For the biomaterial, Poly-L-Lactic Acid (PLLA)—a synthetic polymer that is well characterized in bone regeneration studies—may be employed. [See, Stevens, M. M., Biomaterials for bone tissue engineering. Materials today, 2008. 11(5): p. 18-25; and Kashirina, A., et al., Biopolymers as bone substitutes: A review. Biomaterials science, 2019. 7(10): p. 3961-3983.] However, notwithstanding the use and description of PLLA as a test biomaterial, any kind of implant may be analyzed by the disclosed device/system/method, provided the implant exhibits sufficient strength to be supported by a bolt, and its structural integrity can handle the slight pressure changes in the device.

The materials and the methods of the present disclosure used in one embodiment will be described below. While the embodiment discusses the use of specific compounds and materials, it is to be understood that the present disclosure could employ other suitable compounds or materials. Similar quantities or measurements may be substituted without altering the method embodied below.

### Silicon Wafer Preparation for Photolithography

4-in silicon wafers (University Wafer, Boston, MA, Cat. #452) were carefully washed before being used. The procedure involved washing the wafers with dish soap solution (P&G, Kansas City, KS, Dawn ultra-dishwashing liquid) that has a dilution ratio of 1-to-20. Furthermore, it is important to rub and clean the wafer surface with nonwoven wipes (Texwipe, Kemersville, NC, #TX612 TechniCloth®) in a circular motion for at least 1 min; and then, rinse well with distilled water, using the same circular rubbing motion. Next, the wafers underwent a wet cleaning process by being submerged into an acetone bath for 10 minutes, at 55° C.; and then, in a methanol bath for 5 minutes. The wafers were then removed and rinsed with deionized (DI) water; and dried on a hotplate, at 150° C., for 5 mins (or placed into an oven, at 60° C., overnight). Subsequently, the washed-and-dried silicon wafers were cleaned for the last time via air plasma surface treatment that lasted 30 seconds. Finally, the wafers were spin-coated with Hexamethyldisilazane (HMDS) (Alfa Aesar, Tewksbury, MA, Cat. #999-97-3) to enhance subsequent photoresist adhesion; and dried at 150° C., for 5 seconds.
Spin coating program set up −1) Spread: at 500 rpm for 10 sec with a 100 rpm/s acceleration;
2) Cast: at 2,000 rpm, for 30 sec, with a 500 rpm/s acceleration.

### Master Mold Fabrication

Embodiments of a master molds fabrication of an exemplary in vivo addressable microfluidic device, a sample analysis chip, and a multiplexer chip according to the present disclosure are disclosed below.

In Vivo Addressable Microfluidic Device's Master Molds Fabrication As shown in FIGS. 3A and 3B, an embodiment of an exemplary implanted device may include multiple layers, each of which requires a unique master mold that includes: (1) Control Valve Layer; (2) Flexible Membrane; (3) Payload/Probing Plumbing Layer (with addressable ports); and (4) Sampling Chambers Layer. Initially, AutoCAD files with microscale patterns for each of the layers were printed at 50,800 dpi on 4×4-inch transparency films (Fineline Imaging, Colorado Springs, CO) to generate high-resolution photomasks. The procedures to create the master molds for each layer of the microfluidics device are described below:

Control Valves Layer: The layers were made using the same photolithography procedure: Specifically, SU-8 2075 (Microchem, Westborough, MA, Cat. #Y111074 1000L1GL) photoresist was spin-coated onto wafers at 1,850 rpm, for 40 seconds.
Spin coating program setup −1) Spread: at 500 rpm for 15 sec, with a 150 rpm/s acceleration;
2) Cast: at 1,850 rpm, for 40 sec, with a 200 rpm/s acceleration; 3) Snap-spin to remove edge beads: at 3,400 rpm, for 1 sec, with a 3,400 rpm/s acceleration.
The spin-coated wafers were exposed to UV light (dose: 245 mJ/cm 2); and develop on 4-in silicon wafers, to generate 120 μm high square patterns. The developed photoresist is then fully crosslinked at 180° C., for 2 hours; and then, finally, slowly cooled down to room temperature.

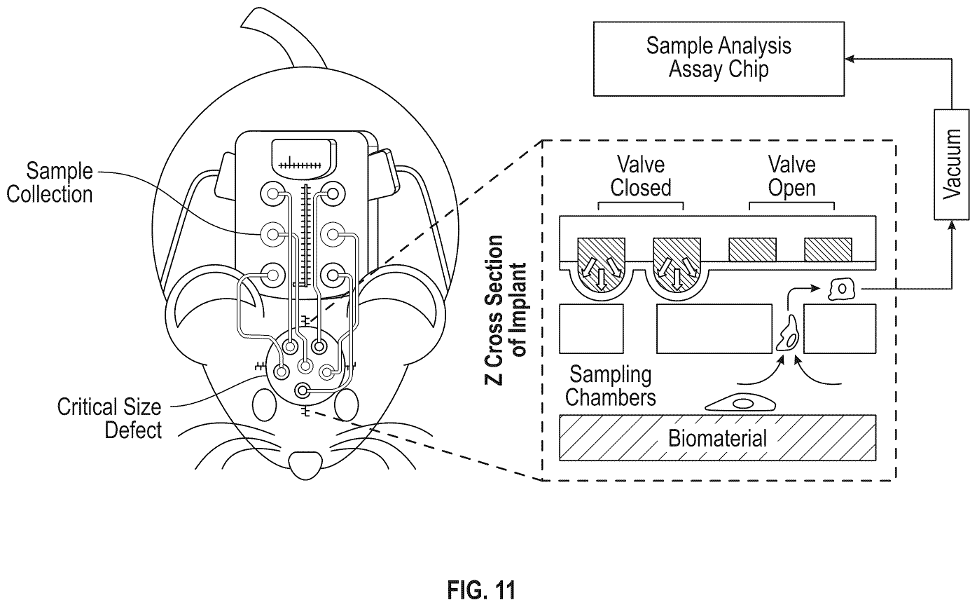

Payload/Probing Plumbing Layer (with addressable ports (e.g., openings connected to channels and operated by valves)): In this embodiment, the master mold for this layer could include two sub-layers (see FIG. 11): (a) Round profile flow channels, created using AZ50 XT positive photoresist (EMD, Somerville, NJ, Cat. #USAW134825), and (b) addressable microfluidics ports on top of the round channels, created using SU8-2150 negative photoresist (Microchem, Westborough, MA, Cat. #Y111077 1000L1GL).

For the Round Profile flow channels Sub-layer, the procedure was adopted from Brower et al. 2017 protocol. [See, Brower, K., A. K. White, and P. M. Fordyce, Multi-step variable height photolithography for valved multilayer microfluidic devices. JoVE (Journal of Visualized Experiments), 2017(119): p. e55276] Specifically, the AZ50 XT was spin-coated on silicon wafers by applying 3 mL of the photoresist to the center of the wafers; and then spin-coated at 1,200 rpm for 40 seconds to form a 55 μm ±2 μm thickness film.
Spin coating program setup −1) Spread: at 200 rpm, for 10 sec, with a 133 rpm/s acceleration; 2)
Cast: at 1,200 rpm, for 40 sec, with a 266 rpm/s acceleration; 3) Snap-spin to remove edge beads: at 3,400 rpm for 1 sec, with a 3,400 rpm/s acceleration.
The spray coating was followed by soft baking of the coated wafer at 110° C. on a programmable hotplate, for 25 mins, starting at 65° C., and with heating ramp rate of 300° C./h. The wafers are then removed from the hotplate, placed in a Petri dish, and left for ambient rehydration for at least 12 hrs, in a dark environment. The wafers were then exposed to 930 mJ/cm 2 of UV light in 17 cycles, with a 30 sec wait time in-between the exposures; and developed immediately afterwards, by immersing in a vessel containing 50 mL of AZ500 k 1:3 developer, at 80° C., for 8-10 min, until the Round Profile features emerge. At this point, the wafers are rinsed with water. The round profiles of the channels were created by baking the wafers at 190° C. on a programmable hotplate, for 15 hours; starting at 65° C., with a heating ramp rate of 10° C./h.

For the Addressable Ports Sub-layer, the SU-8 2150 was spin-coated directly onto the same wafers, at 1,250 rpm, for 1 min.
Spin coating program setup −1) Spread: at 200 rpm, for 30 sec, with a 200 rpm/s acceleration; 2)
Cast: at 1,250 rpm, for 60 sec, with a 200 rpm/s acceleration; 3) Snap-spin to remove edge beads: at 3,400 rpm, for 1 sec, with a 3,400 rpm/s acceleration.
Then, the Round Profiles Sublayer patterns are aligned using a mask aligner [See, Pham, Q. L., et al., A compact low-cost low-maintenance open architecture mask aligner for fabrication of multilayer microfluidics devices. Biomicrofluidics, 2018. 12(4): p. 044119] and exposed to UV (dose: 600 mJ/cm²). After hard baking, the wafers are then mounted on the spincoater and allowed to rotate at 100 rpm. This was followed by spraying the SU-8 developer (Microchem, Westborough, MA, Cat. #Y020100) onto the spinning wafers for 30 seconds-to-1 min, until the addressable port features emerged. Lastly, the developed photoresist was fully crosslinked at 180° C., for 2 hours; then slowly cooled down to room temperature; and ultimately, treated with Perfluorodecyltrichlorosilane (FDTS) (Alfa Aesar, Tewksbury, MA, Cat. #78560-44-8) inside of a vacuum desiccator chamber, for 2 hours.

Sampling Chambers Layer: SU-8 2150 was spin-coated directly on the same wafers, at 1,150 rpm, for 1 min.
Spin coating program setup −1) Spread: at 200 rpm, for 30 sec, with a 200 rpm/s acceleration; 2)
Cast: at 1,150 rpm, for 60 sec, with a 200 rpm/s acceleration; 3) Snap-spin to remove edge beads: at 3,400 rpm, for 1 sec, with a 3,400 rpm/s acceleration.
Then, expose to UV (dose: 600 mJ/cm²); and immerse in a vessel containing the SU-8 developer for 30 minutes, to generate 600 μm-height square features. The developed photoresist was fully crosslinked at 180° C. for 2 hours, and then cooled down slowly to room temperature.

### Sample Analysis Chip's Master Molds Fabrication

The Sample Analysis chip may include three layers: 1) Control Valve Layer; 2) Flexible Membrane; and 3) Sample Analysis layer (with Microwells). The procedures to make the master molds for each layer of the sample analysis chip are similar to the described procedures for the Control Valves Layer and the Payload/Probing Plumbing Layer (with addressable ports).

### Multiplexer Chip's Master Molds Fabrication

The multiplexer chip may include three layers: 1) Control Valve Layer; 2) Flexible Membrane; and 3) Multiplexed Channels with Junction. The procedures to make the master molds for each layer of the sample analysis chip are similar to the procedure for the Control Valves Layer and the Round Profile flow channels Sub-layer.

### Microfluidic Device Fabrication

Embodiments of a fabrication of an in vivo addressable microfluidic device, a sample analysis chip, a multiplexer chip, and a PLLA scaffold are disclosed below.

FIGS. 3A and 3B are diagrams showing how the Payload/Probing Plumbing multilayered mold and the PDMS layers are stacked on top of each other. In FIG. 3A, the master mold for payload/probing plumbing layer (with addressable ports) could include two sub-layers: a round profile flow channels and addressable microfluidics ports on top of the round channels. In FIG. 3B, the in vivo addressable microfluidic device could be created by stacking five different 12 mm diameter PDMS layers and the 8 mm diameter PLLA scaffold on top of each other. 1) Control Valves Layer; 2) 35 μm Flexible Membrane; 3) Payload/Probing Plumbing Layer (with addressable ports); 4) Sampling Chambers Layer; and 5) 8 mm diameter PLLA Scaffold.

### In Vivo Addressable Microfluidic Device

Different Polydimethylsiloxane (PDMS) Sylgard 184 (Dow Corning Corporation, Midland, MI, Cat. #2065622) layers of the devices were generated using soft lithography. The elastomer, with a base-to-agent ratio of 10:1, was poured over the photo-patterned Control Valves Layer mold to reach a thickness of 3 mm. Then, the PDMS casted molds were degassed inside a vacuum desiccator chamber for 2 hours; followed by curing on a hotplate at 65° C., overnight (i.e., 12 hours).

Flexible Membrane (#2 in FIG. 3B) of 35 μm-thickness were created by spin-coating PDMS with a 20:1 base-to-agent ratio onto 4-in silicon wafers at 2500 rpm, for 60 seconds; and then, baked at 65° C., for at least 1 hour.

The Payload/Probing Plumbing and the Sampling Chamber Layers were created by following an established PDMS stenciling procedure. [See, Gao, Y., et al., Digital microfluidic programmable stencil ((DIPS) for protein and cell patterning. RSC advances, 2016. 6(104): p. 101760-101769] Then, all of the layers were peeled off from their master molds; washed with diluted soap; submerged into an acetone bath for 10 minutes, at 55° C.; and then, in a methanol bath for 5 minutes; rinsed with DI water; dried on a 180° C. hotplate; treated with air plasma; and bound to each other, using a PDMS desktop aligner [Li, X., et al., Desktop aligner for fabrication of multilayer microfluidic devices. Rev Sci Instrum, 2015. 86(7): p. 075008], to form one embodiment of a multi-layered microfluidic device as shown in FIG. 3B. Furthermore, during the PDMS bonding process, a biopsy punch (Electron Microscopy Sciences, PA, Cat. #69039-05) with a diameter of 0.5 mm was used to create punch-through inlet and outlet ports for external tubing connections. The order of the single layer bonding to form the multi-layered microfluidic device was as follows: 1) Control Valves Layer; 2) 35 μm Flexible Membrane; 3) Payload/Probing Plumbing Layer (with addressable ports); 4) Sampling Chambers layer; and 5) PLLA scaffold.

### Sample Analysis Chip

PDMS with a base-to-agent ratio of 10:1, was poured over the photo-patterned Control Valves Layer mold and Sample Analysis layer (with Microwells) to reach a thickness of 4 mm and 1.5 mm, respectively. Then, the PDMS casted molds were degassed inside a vacuum desiccator chamber for 2 hours; followed by curing on a hotplate at 65° C., overnight (i.e., 12 hours). The process of making the Flexible Membrane and boding PDMS layers to form the multi-layered microfluidic device are similar to the procedure described in the above section. The order of the single layer bonding to form the multi-layered microfluidic device was as follows: 1) Control Valves Layer; 2) 35 μm Flexible Membrane; 3) Sample Analysis layer (with Microwells); 4) Substrate consisting of a 51×75 mm glass slide (Corning, Corning, NY, Cat. #2947-75X50) to which the device was bound to using air plasma.

### Multiplexer Chip

The process of making the Multiplexer chip is similar to the procedure described in the above section. The order of the single layer bonding to form the multi-layered microfluidic device was as follows: 1) Control Valves Layer; 2) 35 μm Flexible Membrane; 3) Multiplexed Channels with Junction; 4) Substrate consisting of a 51×75 mm glass slide (Corning®, Corning, NY, Cat. #2947-75X50) to which the device was bound to using air plasma.

### PLLA Scaffold Fabrication

The PLLA scaffolds were sketched using AutoCAD to have a donut (or O) shape with an outer diameter of 8 mm, an inner diameter of 1.5 mm, and a 1 mm thickness. They were then printed using an Ultimaker 2+3D printer with the following settings: 0.25 mm nozzle diameter, 0.06 mm layer height, and 80% infill. The printed PLLA scaffolds were then plasma treated for 30 seconds, mineralized by placing in a 0.5 M NaOH solution for 5 minutes, washed with DI water, and placed in an imitated concentrated bodily fluid (10× simulated body fluid (SBF)) for 20 hours (of note, the SBF solution was changed every 2 hours); and finally left in an incubator to dry.

### Results

The disclosed devices, systems and methods implement microfluidics technology to facilitate/support automated, minimally disruptive manipulations of cells and fluids in vivo. These abilities are also “addressable,” meaning that they can be performed independently of each other, at any desired location on a surface of a scaffold in an animal. Of note, the disclosed devices, systems and methods may be used to collect cellular and/or fluidic samples from within the device, and transport them to an external assay chip for ex-situ analysis, thereby overcoming the need for explantation. The nondisruptive nature of such monitoring eliminates the reliance on terminal assays, which in turn, drastically reduces the cost of animal testing, ensures data continuity for multi-time point experiments and reduces the number of animals that need to be sacrificed with ethical benefits.

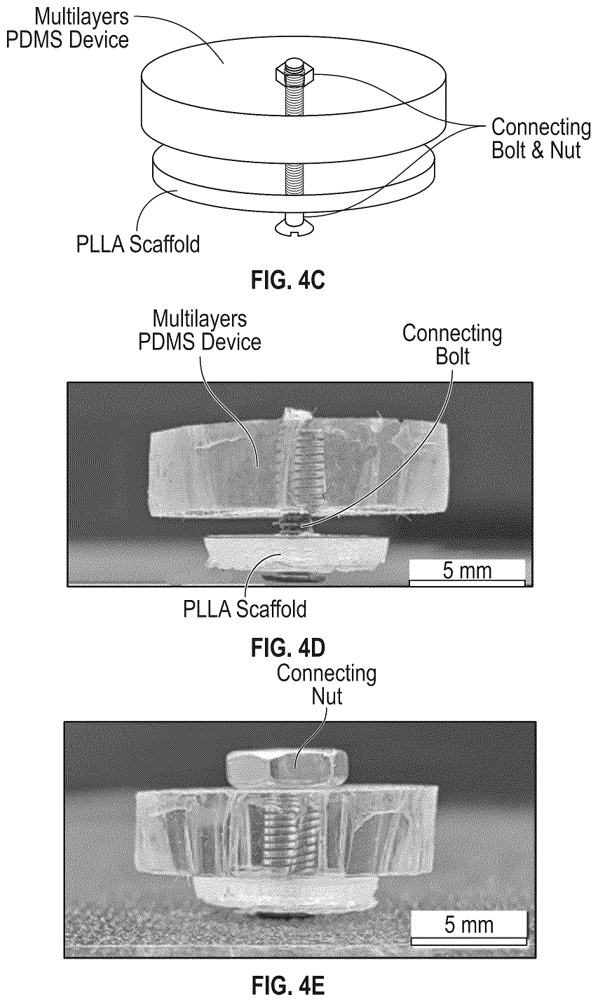

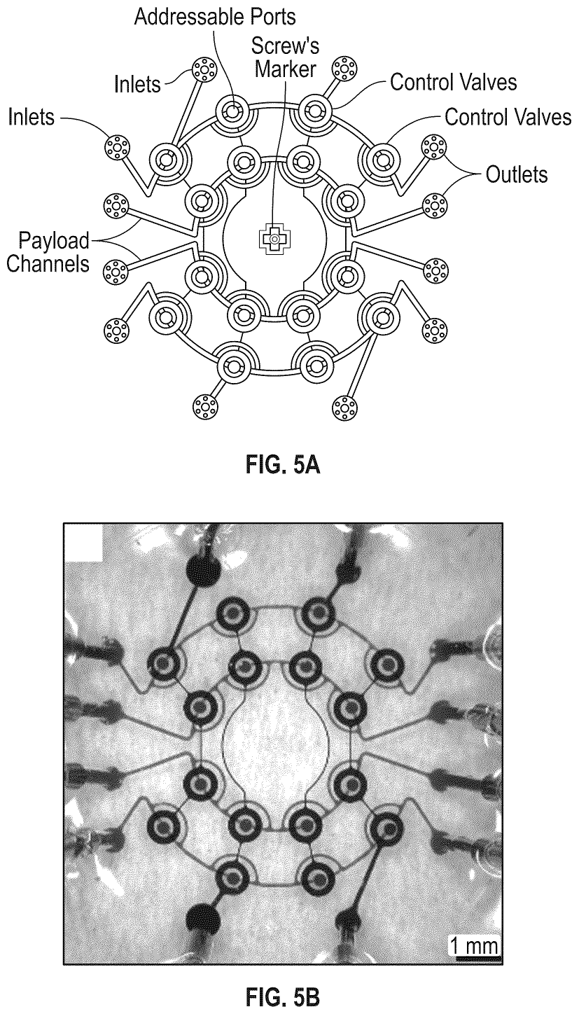

FIGS. 4A-4E show one exemplary embodiment of an addressable microfluidics device made from PDMS for non-destructive analysis of biomaterial implants in vivo according to the present disclosure. The left-hand side of FIG. 4A shows the size of the device relative to a US penny coin, indicating that it is small enough to fit into a critical size calvarial defect in a rat's skull. The tubing on the device may be miniaturized and is thus omitted for clarity from the right-hand side of FIG. 4A, which shows a zoom-in on the device's multilayered microfluidic plumbing. In addition, FIG. 4B shows a circularly-curved grid of addressable ports, which may be fit into an implantable/implanted cylindrical device.

Furthermore, FIGS. 4C, 4D and 4E, show a diagram and photographs of cross-sectional view of a device according to the present disclosure, respectively. Specifically, the noted figures show that the cylindrical PDMS part of the device (i.e., the various layers and sub-layers that have been bonded together) is joined with a scaffold via a small bolt-and-nut. Therefore, a hole is provided in the center of the device through which the bolt is inserted. For example, FIG. 4D shows how a 0.5 cm bolt is used to connect the PDMS device and the PLLA scaffold. It is noted that, in this exemplary embodiment, a flathead bolt is used to support the scaffold from the bottom, to minimize any protrusion into the inner space of the cranium. Furthermore, the PDMS and the PLLA parts of the device in FIG. 4D are intentionally separated from each other on the bolt for demonstration purposes. In contrast, FIG. 4E shows the two parts of the device fully pressed together with a nut on top of the bolt. This is the final assembly/configuration of the exemplary embodiment for implant, e.g., into the skull of an animal.

From FIGS. 4D and 4E, it is also apparent that the bolt was chosen to be as thin as possible (to minimize its interference with the microfluidic plumbing); and that its flat head at the bottom of the device is used to create a mechanical support for the PLLA scaffold. Furthermore, the latter does not add significantly to the overall thickness of the implant, given that the thickness of the rat's skull is about 1 mm. However, the bolt's presence, coupled with the space requirements of the external tubing needle connections, may impact the microfluidic plumbing design, which is constrained by the small diameter of the calvarial defect that the biomaterial implant must fit into. The implications of these challenges are discussed below.

### Modifying the Addressable Microfluidic Plumbing to Encircle the Scaffold Attachment Bolt in a

### Constrained Space of the Calvarial Defect

In exemplary embodiments of the present disclosure, spatial constraints of the calvarial defect model, coupled with its circular shape and with the need to have a bolt in the middle of the device to connect it with the biomaterial scaffold below, influence the microfluidics design. Therefore, in the present disclosure, exemplary embodiments are provided in which the addressable plumbing meet the following requirements: 1) it fits into a limited space of 12 mm diameter (i.e., the device will be slightly wider than the 8 mm implant and will sit between the rat's skull and the skin covering it); 2) it curves around the center hole that fits the bolt for attaching the biomaterial scaffold below the device; 3) the various plumbing inlets and outlets are positioned as far apart from each other as possible to allow space for the numerous tubing insertions (see FIG. 4A) in the constrained area of the device. Of note, if the latter are placed too close to each other, the needles that penetrate the device could create leakages between the neighboring channels and undesired stresses in the PDMS material.

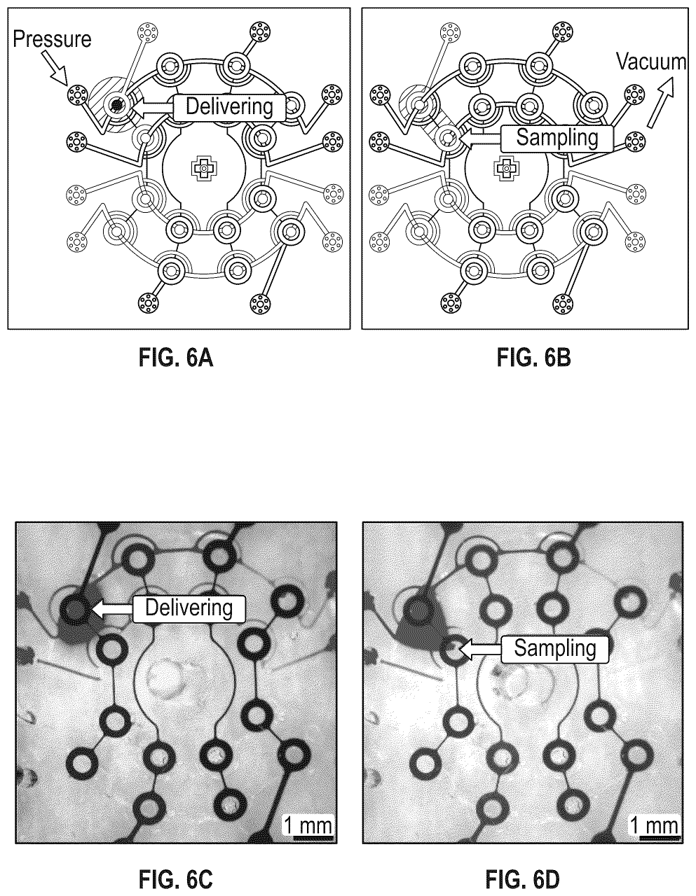

FIG. 5A shows one embodiment of an exemplary plumbing design that satisfies the noted parameters: the Payload/Probing Plumbing Layer is composed of four circularly curved channels, with four addressable ports on each channel; and the Control Valves Layer, which is bonded on top, has four valves, each corresponding to an addressable port, per channel. Also of note, each of the Payload/Probing Plumbing Layer channels requires an inlet and an outlet (making for 8 tubing connections for the layer), but the Control Valves Layer channels only require one inlet/outlet each (i.e., four tubing connections for the layer). Furthermore, in an exemplary embodiment, the latter connections may be placed on alternating sides (i.e., top-bottom-top-bottom) of the device to reduce spatial constrainment of the needles penetrating the PDMS.

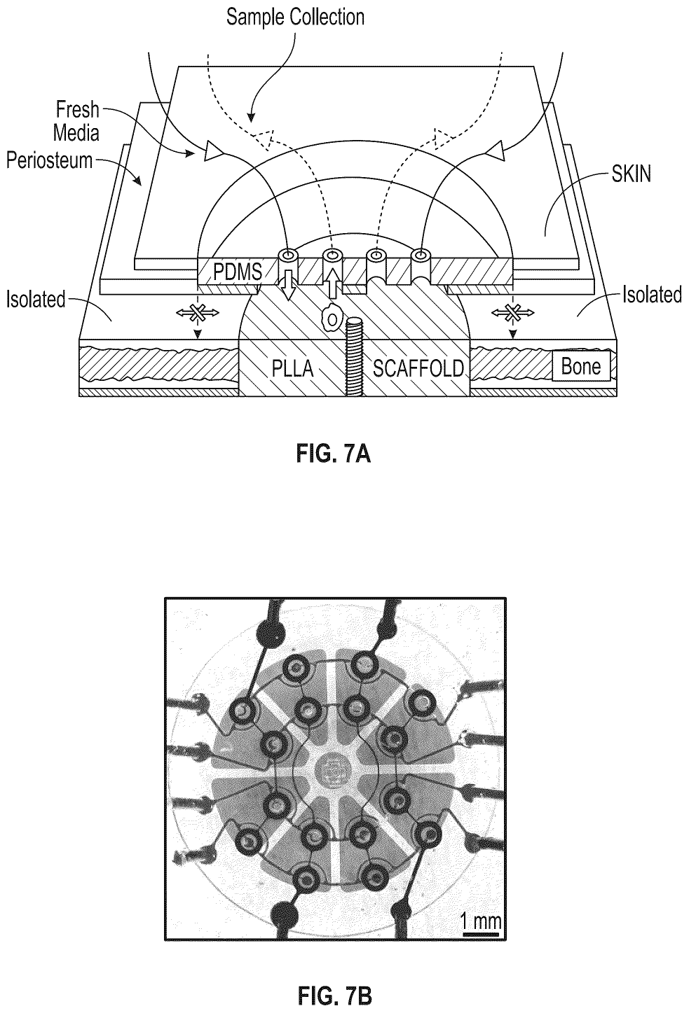

FIG. 5B shows a PDMS prototype of the plumbing design in FIG. 5A. From FIG. 5B, it is apparent that all the channels, the inlets and the external tubing connection needles fit snugly into the 12 mm diameter circular area of the device. Furthermore, FIGS. 6A-6D show that the in vivo implant is capable of effectuating desired fluid and cell manipulation.

The action of delivering and sampling chemicals within the addressable device is shown in the left and right panes of FIGS. 6A/6C and FIGS. 6B/6D, respectively. In the former case, a dye is delivered to the top left corner of the addressable grid of microfluidic ports (see FIGS. 6A & 6C); while in the latter case, the same dye is withdrawn back via a neighboring port in the second row of the addressable matrix (see FIGS. 6B & 6D). In the in vivo setting, the picked-up fluid and/or cells would then be sent off to an external sensor for ex-situ analysis on the SAAC (as in FIG. 2, right pane). This eliminates the need for explantation of the scaffold, ensuring continuous monitoring of the biology occurring during bone regeneration within the animal's native tissue environment. Furthermore, this can be done continuously over long periods, given that the entire process is automated and as such, does not require any human involvement.

However, the actions of either delivering or withdrawing fluids within the implanted device can result in a pressure imbalance in the host's head, which can disrupt the experiment by either breaking the biomaterial scaffold that is being analyzed and/or presenting a danger to the animal by upsetting its intracranial equilibrium. Therefore, disclosed below are some preventative measures in the present design to address/obviate these risks.

Maintaining Cranial Fluid Equilibrium and Preventing Contamination from the Surrounding Tissues via Sampling Chambers

An additional implication of implanting a microfluidics device in the cranium of a living animal is its potential interaction with the host's body, which is not desired. For example, some potential issues are: 1) fluid payloads delivered to the device could leak out into the host's surrounding tissues, potentially upsetting the intracranial fluid pressure in the animal's head and potentially hurting or even killing the host; 2) a reverse situation could occur where a mix of the host's bodily fluid/cells from the areas surrounding the implant could contaminate the probings collected from the Sampling Chambers of the device; and/or 3) if excess vacuum or pressure are created in the device, those conditions could cause the scaffold to collapse or break, thereby jeopardizing the experiment and endangering the animal's well-being.

Therefore, it is desirable to: a) isolate the microfluidics portion of the device from the host's physiology, and b) maintain a fluid equilibrium inside of its chambers, such that no excess pressure or vacuum are created by the performed plumbing manipulations. In other words, if any fluid flows into the device, an equal amount is desirably removed from it somewhere else simultaneously and vice versa.

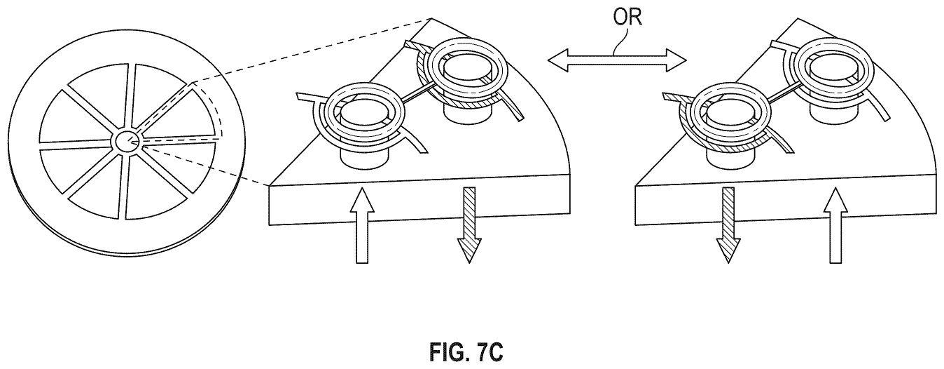

To address these issues, a Sampling Chambers Layer is provided in exemplary embodiments of the present disclosure, situated between the PDMS part of the device and the biomaterial scaffold (see Layer #4 in FIG. 3B, and also see FIG. 7A). The Sampling Chambers Layer serves at least two purposes: 1) The thick circular band, along the circumference of the layer, serves as a “wall” that sits on top of the skull bone (attached with fibrin glue) and isolates the device's microfluidic plumbing from the interstitial fluids of the host's body (see FIG. 7A); and 2) The area inside of the circle is broken up into a plurality (e.g., eight) of identical “pizza slice”-shaped chambers (see FIG. 7B), which effectively divide the surface of the implanted scaffold into separate sampling regions.

Inclusion of the Sampling Chambers Layer is beneficial because it: a) reduces diffusion between the neighboring regions, thereby increasing the spatial accuracy (i.e., “resolution”) of the probing; and b) isolates two addressable microfluidic ports per each “pizza slice”-shaped chamber (see FIG. 7C). The isolation aspect of the design serves a fluid pressure balancing function because, while one port is active (i.e., delivering or sampling fluid), the other port in the chamber may be kept open, thereby preventing any pressure or vacuum build-up that could potentially lead to collapse of the PDMS device; or to the deformation/cracking of the PLLA scaffold and/or causing brain damage in the animal/host, due to the strong pressure/vacuum forces transmitted through the PLLA's pores. For example, when one port is open to a vacuum line to collect a sample, the neighboring port would be open to a fresh media source to quench the negative pressure in the same chamber (see FIGS. 7A & 7C).

As the Probings are Collected, they May be Sent Off to a SAAC for Analysis.

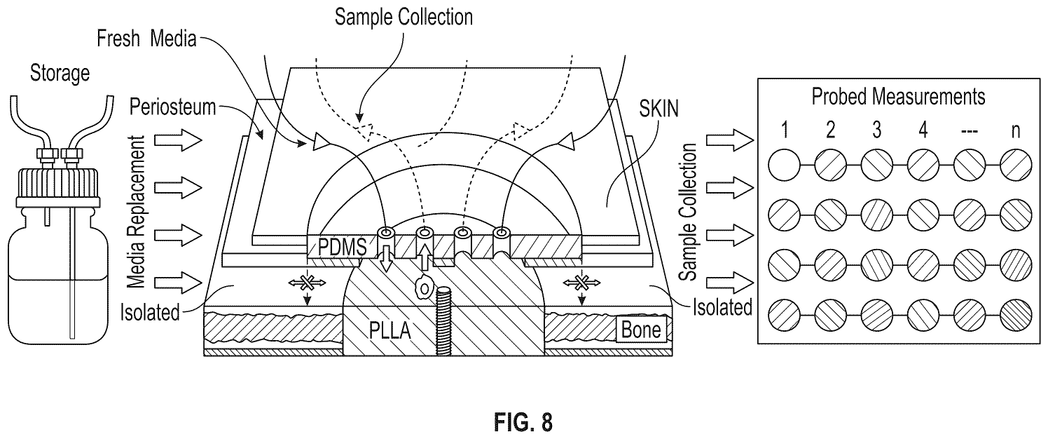

Storing and Analyzing Collected In Vivo Samples on an Ex Vivo Assay Chip As the probings are collected within the microfluidic implant, the pressure balance within it is desirably maintained. For this reason, the implant could be connected to a Media Replacement Storage (see FIG. 8, left pane), which may provide the fluids necessary to substitute the volumes subtracted by the probings. In other words, the microfluidic device will draw the replenishment media from this storage as it is collecting samples from the “pizza slice”-shaped Sampling Chambers (see FIG. 8, center pane), to maintain the net amount of fluid in the device constant (or substantially constant). Once the probings are obtained, they are generally flowed out of the implant and into an ex vivo SAAC (see FIG. 8, right pane), thereby overcoming the need for sample explantation.

When on the chip, the collected probings can be either stored to be subsequently removed by an operator for off-line analysis; or on-chip assays can be performed directly in the storage wells. For the latter option, some potential/illustrative chemical tests that are appropriate for the calvarial defect bone tissue engineering application are discussed below, while an exemplary design of the chip is discussed herein.

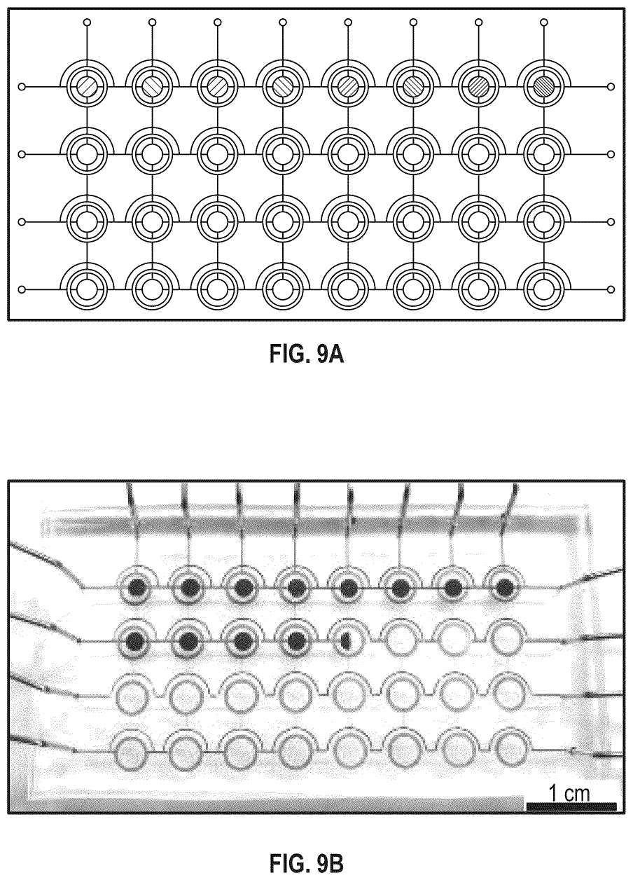

The chip's plumbing may be a microfluidic analogue of a conventional well plate (e.g., 96 well plate), with each of the storage compartments being individually addressable (see FIGS. 9A and 9B). Exemplary designs are described in the literature, see, e.g., Gao, Y., et al., Digital microfluidic programmable stencil (dMPS) for protein and cell patterning. RSC advances, 2016. 6(104): p. 101760-101769; Wang, H.-Y., N. Bao, and C. Lu, A microfluidic cell array with individually addressable culture chambers. Biosensors and Bioelectronics, 2008. 24(4): p. 613-617. In implementing a design, an appropriate number of rows and columns for the well matrix is selected. Of note, given that the dimensions of the chip are not limited by in vivo size constraints, the chip can be essentially any size, because as many (or as few) probings as needed can be processed by the chip at any desired frequency.

In an exemplary design, the chip includes four rows of wells to match the number of Payload/Probing Plumbing channel rows in FIGS. 5A and 5B, and in FIGS. 6A-6E, because this reduces the plumbing complexity. Meanwhile, the chip's number of columns remains a free parameter, which would be determined based on the number of the desired time points, locations and/or signals that the user wants to measure (see, e.g., FIG. 9, right pane).

For example, a single time point measurement at all 16 addressable port locations in the implant would require four columns (i.e., a 4×4 matrix of wells); while storing two time points would require eight columns (i.e., a 4×8 matrix of wells); and so on. Alternatively, if an entire “pizza slice”-shaped sampling chamber is considered to be a single “location” (since the addressable ports work in tandem pairs, as shown in FIG. 7C), then the number of the wells per time point would be halved (i.e., 2 chamber rows×4 chamber columns translates to a requirement of a total of 8 storage wells). Conversely, to measure multiple biological signals per time point at each location, the number of total wells from the examples above would be multiplied by however many signals are being detected. For example, to measure four different biological signals per a sampling chamber, this would translate to a chip with a total of 32 wells.

In an exemplary implementation, a SAAC with a 4×8 addressable grid of storage wells is schematically depicted (see FIG. 9A). FIG. 9B shows a working prototype of a micro-well array as shown in FIG. 9A.

### Reducing the External Equipment Requirement via a Combinatorial Multiplexer Chip

Given that the addressable microfluidic plumbing requires a considerable amount of external hardware equipment (e.g., pneumatic solenoid valves, pressure regulators, gauges, etc.) to run, managing two such chips (i.e., the Addressable Implant and the SAAC) can become costly and complicated. Furthermore, if many locations, time points and/or biological signals need to be measured between the SAAC replacements, then the growing size of the latter will increase that requirement even further. To put it into perspective, the 4×4 implantable device described above uses a manifold of 8 solenoids—the costliest components in the system—to actuate its 4 control valve channels and 4 flow channels.

Moreover, the cost accumulates further for the hardware needed to run the SAAC: although its rows can be run by the same solenoids as those actuating the implant's Payload/Probing Plumbing channels, because they are basically shared between the two chips, its columns do require extra solenoids to operate. So, for a typical 3-day experiment, with a 4-hour probing frequency (i.e., 6 times per day), the number of the columns in the SAAC that detects four biological signals per implant (as in FIGS. 9A and 9B) would have to be increased from 8 to 3×6×8=144. That is, the solenoid cost of running a SAAC of this size would increase by a factor of 18 with associated tubing/wiring complexity.

A Combinatorial Multiplexer makes it possible to reduce the external hardware requirements (albeit at a penalty of increasing the complexity of the on-chip's plumbing). Namely, due to its N!/(N/2)!² scaling, up to 252 lines can be actuated with just 10 control lines (i.e., with only 10 solenoids). This means that by adding a multiplexer to flow systems as disclosed herein, the equipment cost of running the 144 column SAAC in the example above can be reduced by a factor of 10.

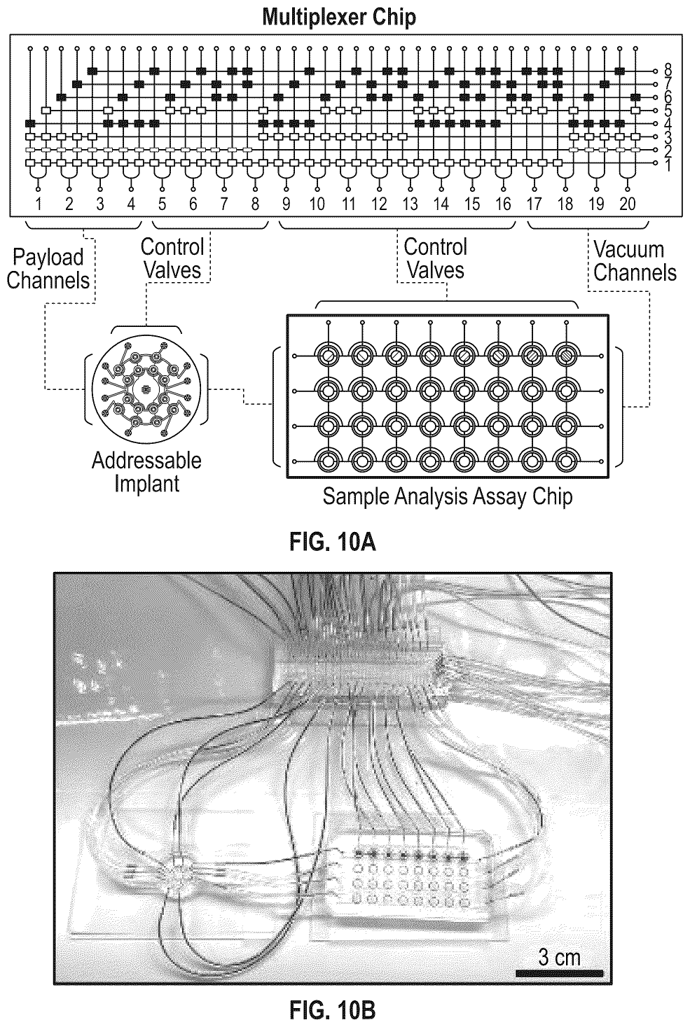

FIGS. 10A and 10B show one embodiment of an exemplary integrated system, with all three of the microfluidics chips connected: the Addressable Implant, the SAAC, and the Combinatorial Multiplexer. For the latter, an 8 control lines version may be employed (multiplexer rows in FIG. 10A corresponding to horizontal tubing entering the multiplexer from the left side in FIG. 10B), since it can actuate up to a total 70 flow lines (multiplexer columns prior to Junctions in FIG. 10A). Of note, the system as described in FIG. 7B and in FIGS. 9A/9B requires less than that to run. In fact, multiplexing just 40 flow lines is sufficient for operating the Addressable Implant and the SAAC described above, respectively. Therefore, the remaining 30 unused flow lines of the multiplexer need not be fabricated (and are thus not shown in FIGS. 10A/10B).

As far as the 40 flow lines that are used, they are merged into pairs (see the 20 numbered Junctions in FIG. 10A). For example, the Junctions #1-4 are dedicated for multiplexing the inputs of the payload/probing plumbing on the Addressable Implant. Each of the junctions allows the system to alternate between two different fluid and/or pressure source inputs, while combining them into a single multiplexed output. For example, one of the two lines belonging to the Junction #1 may be active, while the other is not. The multiplexer can alternate between them to flow fluids containing different drugs, media, etc., through the Payload Channel resulting from the Junction #1 Likewise, it can also alternate between pressurized and non-pressurized lines that belong to Control Valve Junctions #5-8, to either inflate or deflate the O-shaped valves on the Addressable Implant. Similarly, the Junctions #9-16 in FIGS. 10A/10B are used to either inflate or deflate the addressable O-shaped valves on the SAAC. Lastly, the Junctions #17-20 are used in tandem with the first four junctions to flow the payloads/samples through the four rows in the Addressable Implant and SAACs. However, in the case of the former junctions, the multiplexer alternates between non-pressurized and vacuum lines. The negative pressure, in turn, helps to pull the payloads and/or samples through the two chips.

An example of the multiplexer's operation is also shown in FIG. 10A: by actuating its Control Lines #4 and 6-8, the multiplexer allows for flow to only go through the right arm of the multiplexed Junction #1, while the rest of the junctions are fully blocked. Similarly, the multiplexer can activate or disable the inflatable O-shaped valves on the Addressable Implant and the SAAC to achieve the addressable manipulation at the targeted microfluidics ports or wells, respectively. To that end, FIG. 10B shows an exemplary system using the multiplexer to fill the top row of the SAAC with a dye.

Table 1 shows how the choice of a different-sized multiplexer affects the total number of time points and/or biological signals (see the last column in Table 1) that can be stored/analyzed on the SAAC. For these calculations, it is assumed that the implantable device's matrix of addressable ports remains at the fixed size of 4×4 presented herein, which takes 8 payload/sampling lines (arms of the Junctions #1-4 in FIG. 10A), 8 control valve lines (i.e., arms of the Junctions #5-8 in FIG. 10A) to operate and 8 vacuum lines to operate (i.e., arms of the Junctions #17-20 in FIG. 10A). Of note, the design for the addressable implant may be implemented such that a fixed number of 22 multiplexed channels are used for the implant payload channels, the implant control channels, and the sample chip vacuum channels. Also note that the number of Control Lines can only be an even number for a combinatorial multiplexer.

TABLE 1
Scaling capacity of the multiplexer plumbing
assuming a 4 × 4 Addressable Implant.

| | | No. of | Remaining | Total | Total |
|---|---|---|---|---|---|
| | Total | remaining | possible | possible | possible |
| | possible | possible | multi- | SAAC | time |
| No. of | multi- | multi- | plexed | microwells | points |
| Control | plexed | plexed | junctions | (4 × n | and/or |
| Lines | channels | channels | (n) | array) | signals |
| 8 | 70 | 46 | 23 | 92 | 5 (11) |
| 10 | 252 | 228 | 114 | 456 | 28 |
| 12 | 924 | 900 | 450 | 1800 | 112 |
| 14 | 3432 | 2410 | 1205 | 6820 | 426 |
| 16 | 12870 | 12848 | 6424 | 25696 | 1606 |

To illustrate how the data in Table 1 is calculated, an example for calculation of the first row is provided. Here, an 8-control line version of the combinatorial multiplexer is examined. Its theoretical maximum is the actuation of 70 flow lines, as per the formula:

N!/(N/2)!², where N=8.

Out of these 70 flow lines, 24 must be dedicated to operating the Addressable Implant chip. So: 70 (total possible multiplexed flow lines) −8 (implant payload channels) −8 (implant control channels) −8 (sample chip vacuum channels)=46 (remaining possible multiplexed channels)

Next, it is noted that it takes two flow lines to operate a single addressable microwell column on the SAAC, because pressurized and non-pressurized lines must be merged together into a single Control Valve channel (see the formation of Junctions #9-16 in FIG. 10A). Therefore, the number of total possible addressable microwell columns on the SAAC is equal to half of the remaining possible multiplexed channels:

46 (remaining possible multiplexed channels)/2 (multiplexed channels per junction)=23 (remaining possible multiplexed junctions) which translates to 23 (total possible microwell columns)

Next, the number of columns on the SAAC translates to the number of microwells. Based on an assumption that the Addressable Implant chip has 4 payload/sample flow channel rows, this means that the number of SAAC wells is quadruple the number of columns (because the row channels are shared between the two chips):

4 (implant channel rows)*23 (total possible microwell columns)=92 (total possible SAAC microwells)

Lastly, assuming that the user wants to store/analyze every location in the 4×4 addressable matrix, each time point or biological signal that is to be measured would consume 16 (or 8, if each Sampling Chamber is treated as a “location”) SAAC microwells:

92 (total possible SAAC Microwells)/16 (implant probing locations)=5.75 (time points and/or signals)

Given that the final calculated value is not a whole number, the value may be rounded down to the nearest integer to conclude that 5 (or 11, if each Sampling Chamber is treated as a “location”) total time points and/or biological signals that could be stored/analyzed on the SAAC.

The disclosed systems and methods have a wide range of potential applications. By way of illustration, a bone tissue engineering use case is described herein to illustrate the utility thereof. Specifically, an exemplary device has been demonstrated for implantation in an 8 mm diameter critical size defect in a rat's skull. As noted above, this is a “gold standard” model for testing engineered bone tissue scaffolds in living animals. For demonstration purposes, the well-characterized PLLA biomaterial is utilized, although any kind of implant could be implemented for purposes of the disclosed device/system/method (provided it is not too soft to be supported by a bolt, and its structural integrity can handle the slight pressure changes in the device).

Accounting for External Tubing and Wiring during the Implantation of the Microfluidics Device into a Living Animal

Based on prior implantations of artificial devices into the heads of living animals, e.g., in microdialysis experiments, it is reasonable to expect that the disclosed devices/systems too can be tolerated by host animals, e.g., rats, given that it the device/system would not invade as deeply into their craniums. Of note, when implanting the disclosed device/system, like conventional microdialysis probes, external hardware components, such as wiring and tubing, protrudes from the animal's head permanently after the surgery (as shown in FIG. 1B). Therefore, the calvarial defect protocol should take account for the implanted microfluidics device being connected to external hardware components. For example, the entire device assembly could be glued to the animal's skull using dental acrylic. After that, the periosteum subcutaneous tissue would be placed over the resin, to improve adherence; and sutured using appropriate suture material. The skin would then be similarly sutured. Finally, the tubing protruding from the top of the head amy be glued in place using the dental acrylic.

Assessing the Biomaterial Implant's Performance using Ex Vivo ELISA Assays of the In Vivo Fluid Samples

Given that most of the probings will be secretions by the cells (and not the cells themselves), histology of the samples collected from the in vivo environment is implausible. Sandwich ELISA assays may be used for analyzing the biomaterial implant's performance in vivo. Specifically, they can be used to quantify the tissue in-growth from the host's body based on the following common bone-specific markers:

Differentiation: Osteogenic commitment of stem cells (responsible for new tissue synthesis) can be monitored via Sandwich ELISA assays that analyze the collected liquid for the secretion of (BMP-2) and Osteocalcin (OC)— bone-specific proteins synthesized by osteoblast bone cells.

Extra Cellular Matrix (ECM) Synthesis: bone-specific alkaline phosphatase (BAP) and procollagen type I carboxy-terminal propeptide (PICP) (an indicator of bone type I Collagen) ELISA can be used to monitor the synthesis of the “hard” (i.e., calcified) and “Soft” (primarily Collagen I) ECM formation, respectively.

Table 2 summarizes four proposed ELISA signal readouts for an exemplary engineered bone implant analysis application.

TABLE 2
Summary of possible sandwich ELISA assays
for bone regeneration analysis.

| Monitored Tissue Regeneration | Secretion Assays |
|---|---|
| Differentiation to Osteoblast Lineage | BMP-2 [Zhang, Y., et al., |
| | Patterning osteogenesis |
| | by inducible gene |
| | expression in microfluidic |
| | culture systems. |
| | Integrative Biology, 2010. |
| | 3(1): p. 39-47[22]] |
| | OC [Nakamura, A., et al., |
| | Osteocalcin secretion as |
| | an early marker of in vitro |
| | osteogenic differentiation |
| | of rat mesenchymal stem |
| | cells. Tissue Engineering |
| | Part C: Methods, 2009. |
| | 15(2): p. 169-180.] |
| Bone Extra Cellular Matrix (ECM) | Hard ECM: BAP |
| | [Roudsari, J.M. and S. |
| | Mahjoub, Quantification |
| | and comparison of bone- |
| | specific alkaline |
| | phosphatase with two |
| | methods in normal and |
| | paget's specimens. |
| | Caspian journal of internal |
| | medicine, 2012. 3(3): p. |
| | 478; Golub, E.E. and K. |
| | Boesze-Battaglia, The role |
| | of alkaline phosphatase in |
| | mineralization. Current |
| | opinion in Orthopaedics, |
| | 2007. 18(5): p. 444-448] |
| | Soft ECM: PICP [Seo, |
| | W.-Y., et al., Production |
| | of recombinant human |
| | procollagen type I C- |
| | terminal propeptide and |
| | establishment of a |
| | sandwich ELISA for |
| | quantification. Scientific |
| | reports, 2017. 7(1): p. |
| | 15946] |

Any use of the foregoing the ELISA assays should be validated for use in analyzing in vivo bone formation in real time prior to implementation in a clinical testing. This validation can be undertaken in a variety of ways. For example, by comparing the ELISA results to literature data that quantifies bone regeneration in PLLA implants via conventional microscopy assays (e.g., ALP, Alizarin Red, proteoglycans, etc.). Alternatively, validation can be undertaken by explanting the scaffolds at the very end of the experiment (i.e., when the animal is euthanized), followed by conventional staining assays/histology performed on the retrieved samples to check for a confirmation of the bone formation and for an absence of collagen signals. Although not real-time, these measurements would provide more confidence to device read-outs using more traditional approaches.

### Miniaturizing Supporting Hardware to Make the Device Completely Wearable by the Animal

Having a live animal being permanently connected to external hardware can present a problem with it attempting to detach itself, and in the process dislodging and/or damaging the implanted device. Hence, the external hardware is ideally miniaturized to the extent possible, e.g., so that the hardware can be fitted into a “backpack” worn by the host/rodent (see, e.g., FIG. 11, left pane). For example, the programmable logic controller/house air setup currently operating the present device can be substituted by, for example, an iPRECIO® SMP-200 programmable micro infusion pump, which includes the following design characteristics: 1) 6 months battery life; 2) 147 g weight; 3) 12×6.7×3.5 cm size; 4) wireless communications. Additionally, the external solenoid valves may take the form of the VHS Series miniature solenoid valves made by The Lee Company. The noted solenoids were designed for applications requiring precise dispense volumes in the microliter to nanoliter range and have a weight of just 1.8 g and a size of 2 cm length×0.5 cm diameter.

A miniature power source, like a watch battery, may be included into the assembly, e.g., the “backpack”. Additionally, the readings of the ELISA measurements could be done on the SAAC automatically. This can be accomplished based on O₂ concentration generated from an enzymatic digestion of H₂O₂ substrate (pre-loaded into the microwells) [see, e.g., Liu, D., et al., A fully integrated distance readout ELISA-Chip for point-of-care testing with sample-in-answer-out capability. Biosensors and Bioelectronics, 2017. 96: p. 332-338.]. Once the gas is produced, it would displace a dye into a microfluidics read-out channel with markings that correspond to different pressures (similar to how temperature is read from the gradations of a mercury thermometer). These values will then be indicative of the bone generation markers in the implant at each time of the probes' collections. Furthermore, the reading can be done by using a small LED light+ sensor combination, and possibly even transmitted wirelessly using a miniature Wi-Fi module. And the latter could also be used for sending command signals to the system remotely.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A method for non-destructive in vivo analysis of implanted materials, comprising:
a. inserting a microfluidics device within a living organism,
b. collecting samples from the living organism at a plurality of distinct targeted locations, wherein the samples comprise fluid samples, biological samples or both fluid and biological samples, and
c. exporting the collected samples out of the living organism for analysis ex vivo.

2. The method of claim 1, wherein the samples are collected continuously over time.

3. The method of claim 1, wherein the samples are collected at pre-set intervals.

4. The method of claim 1, wherein the microfluidics device includes:
a. a control valves layer,
b. a flexible membrane positioned beneath the control valve layer,
c. a payload plumbing layer with customizable addressable ports under the flexible membrane, and
d. a layer of chambers beneath the payload plumbing layer from which the samples are collected.

5. The method of claim 4, wherein the microfluidics device comprises at least one programmable addressable valve capable of regulating fluid flow.

6. The method of claim 4, wherein the microfluidics device further comprises an integrated multiplexer.

7. The method of claim 6, wherein the integrated multiplexer is configured to function as a fluidic switchboard by directing flow to and from the microfluidic device.

8. The method of claim 7, wherein the integrated multiplexer directs flow to and from the microfluidics device based on analytical need.

9. e method of claim 6, wherein the integrated multiplexer reduces external hardware requirements for analysis of the samples.

10. The method of claim 4, where the microfluidics device includes an integrated analysis chip that processes the collected samples.

11. The method of claim 4, wherein the microfluidics device is configured and dimensioned for insertion into a cranial bone defect.

12. The method of claim 1, further including removal of the microfluidics device from the host organism for post-analysis.

13. The method of claim 4, where the sampling chambers layer is configured to isolate the microfluidics device from surrounding bodily fluids of the living organism.

14. The method of claim 1, wherein the microfluidics device further comprises means for maintaining fluid equilibrium within the living organism during sample collection.

15. The method of claim 1, further comprising using the ex vivo analysis of the samples to monitor osteogenesis within the living organism.

16. The method of claim 1, further comprising integrating the microfluidics device with a structural scaffold fabricated at least in part from a material being tested.

17. The method of claim 16, wherein the microfluidics device is attached to the structural scaffold by a central fastening mechanism.

18. The method of claim 1, wherein the microfluidics device defines a curvature to conform to one or more anatomical structures of the living organism.

19. The method of claim 1, further comprising leveling the microfluidics device when implanted in the living organism.

20. The method of claim 4, wherein the sampling chambers layer includes an outer circular boundary and an inner section divided into multiple segments for distinct sampling.

21. The method of claim 1, wherein the sample analysis is conducted using an assay chip.

22. A microfluidics system, comprising:
a. a control valves layer;
b. a flexible membrane beneath the control valve layer;
c. a payload plumbing layer with configurable addressable ports beneath the flexible membrane;
d. a sampling chambers layer beneath the payload plumbing layer;
e. an integrated multiplexer for fluid control; and
f. an integrated analysis chip for sample analysis.

23. The microfluidics system of claim 22, wherein one or more of the control valves layer, the payload plumbing layer and the sampling chambers layer is independently prepared and then joined together with the flexible membrane.

24. The microfluidics system of claim 23, wherein one or more of the control valves layer, the payload plumbing layer and the sampling chambers layer is fabricated by a fabrication method selected from the group consisting of lithography, etching, machining laser-cutting, 3-D printing, and combinations thereof.

25. The microfluidics system of claim 22, wherein the control valves layer, the payload plumbing layer, the sampling chambers layer and the flexible membrane are joined together by a bonding method selected from the group consisting of chemical bonding, thermal bonding, pressure bonding, and combinations thereof.

26. The microfluidics system of claim 22, wherein the control valves layer, the payload plumbing layer and the sampling chambers layer are manufactured in their entireties by a method selected from 3-D printing, sacrificial template gel-casting, or a combination thereof.

27. The microfluidics system of claim 22, further comprising a system for analysis of biological processes, comprising: (i) a data processing unit configured to analyze data obtained from collected samples; and (ii) communication means for transmitting data to the data processing unit.

28. The microfluidics system of claim 22, further comprising a plurality of chemical assays tailored for analyzing samples gathered in vivo.

29. The microfluidics system of claim 28, wherein the plurality of chemical assays includes one or more assays for analyzing cranial bone defect-related properties.

30. The microfluidics system of claim 22, wherein (i) the control valves layer, (ii) the flexible membrane, (iii) the payload plumbing layer, (iv) the sampling chambers layer, (v) the integrated multiplexer, and (vi) the integrated analysis chip, are miniaturized and integrated into a single portable system.
